(19) United States
(12) Patent Application Publication
(10) Pub. No.: US 2023/0285720 A1
(43) Pub. Date: Sep. 14, 2023

(54) CATHETER AND PRODUCTION METHOD THEREFOR

(71) Applicant: ASAHI INTECC CO., LTD., Seto-shi (JP)

(72) Inventor: Soushi ISOGAI, Seto-shi (JP)

(73) Assignee: ASAHI INTECC CO., LTD., Seto-shi (JP)

(21) Appl. No.: 18/140,390

(22) Filed: Apr. 27, 2023

Related U.S. Application Data
(63) Continuation of application No. PCT/JP2020/040910, filed on Oct. 30, 2020.

(51) Int. Cl. A61M 25/01; A61M 25/00

(52) U.S. Cl. CPC A61M 25/0108; A61M 25/005; A61M 25/09

(57) ABSTRACT

A catheter comprises a tubular body having a lumen, a reinforcing body provided on an outer periphery of the tubular body, and a radiopaque marker member disposed along a circumferential direction of the reinforcing body. In the longitudinal axis direction of the tubular body, a distal end of the reinforcing body is positioned at the same site as a distal end of the marker member, or at a site further toward a distal end side. A distal end portion of the marker member is joined to the reinforcing body, and a portion other than the distal end portion of the marker member is not joined to the reinforcing body.

### CROSS REFERENCE TO RELATED APPLICATION

This application is a bypass continuation application of International Application No. PCT/JP2020/040910, filed Oct. 30, 2020, the content of which is incorporated herein by reference in its entirety.

### BACKGROUND

The disclosed embodiments relate to a catheter and a production method therefor.

For example, when treating a lesion or the like that has formed inside a blood vessel, a long hollow catheter is used as a medical device that is inserted into the blood vessel.

Such a catheter is required to allow the position of the catheter to be accurately grasped inside the blood vessel so that another medical device or the catheter itself can be safely and reliably guided to the lesion inside the blood vessel.

As such a catheter that enables the position inside a blood vessel to be grasped, for example, proposed is a technique that provides an X-ray opaque marker at a specific site of a tubular shaft, and enables the position of the catheter to be grasped in an X-ray transmission image, according to JP 2004-298361.

According to the technique above, for example, a coil-shaped member formed of a metal wire is used as the X-ray opaque marker, and the marker is fixed by crimping the marker to the outer peripheral surface of a specific site of the tubular shaft.

### SUMMARY

However, in the conventional technique described above, when crimping the marker, the entire marker is firmly fixed to the tubular shaft along the longitudinal axis direction. As a result, the crimping increases the rigidity (decreases the flexibility) of the tubular shaft, and there is a concern that the operability of the catheter will decrease, especially in a curved blood vessel.

The disclosed embodiments have been made based on the above circumstances, and an object thereof is to provide a catheter and a production method therefor that enable a marker member to be fixed to a reinforcing body while ensuring adequate flexibility.

To achieve the above object, some embodiments of the present disclosure include a catheter including: a tubular body having a lumen; a reinforcing body provided on an outer periphery of the tubular body; and a radiopaque marker member disposed on the reinforcing body along a circumferential direction of the reinforcing body; in which in a longitudinal axis direction of the tubular body, a distal end of the reinforcing body is either positioned at the same site as a distal end of the marker member, or positioned at a site further toward a distal end side than the distal end of the marker member, and a distal end portion of the marker member is joined to the reinforcing body, and a site of the marker member excluding the distal end portion of the marker member is not joined to the reinforcing body, and
a production method of a catheter provided with a tubular body having a lumen, a reinforcing body provided on an outer periphery of the tubular body, and a radiopaque marker member disposed on the reinforcing body along a circumferential direction of the reinforcing body, the method including: disposing, in a longitudinal axis direction of the tubular body, a distal end of the reinforcing body at either the same site as a distal end of the marker member, or at a site further toward a distal end side than the distal end of the marker member; and welding a contact portion between the reinforcing body and the distal end portion of the marker member without applying a mechanical external force onto the contact portion, and without welding a site of the marker member excluding the distal end portion of the marker member.

### BRIEF DESCRIPTION OF THE DRAWINGS

FIG. 1 is a schematic side view showing an embodiment of the disclosed embodiments.

FIG. 2 is an enlarged schematic side view showing a part of a catheter in

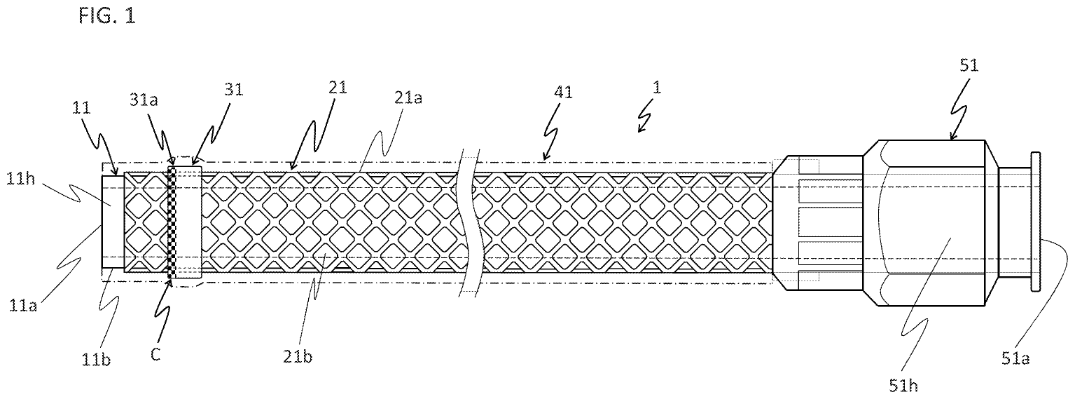

FIG. 1.

FIG. 3 is an enlarged schematic side view showing a part of an embodiment of the disclosed embodiments.

FIG. 4 is an enlarged schematic side view showing a part of an embodiment of the disclosed embodiments.

FIG. 5 is an enlarged schematic side view showing a part of an embodiment of the disclosed embodiments.

FIG. 6A is a schematic side view of an embodiment of the disclosed embodiments, showing a state immediately after the reinforcing body is disposed on the outer peripheral surface of the tubular body.

FIG. 6B is a schematic side view of a catheter in FIG. 6A, showing a state immediately after the marker member is disposed on the reinforcing body.

FIG. 6C is a schematic side view of the catheter in FIG. 6A, showing a state where laser light is irradiating the distal end portion of the marker member.

FIG. 6D is a schematic side view of the catheter in FIG. 6A, showing a state where the tubular body and the covering member have been welded together.

FIG. 7A is a schematic side view of an embodiment of the disclosed embodiments, showing a state where laser light is irradiating the distal end portion of the marker member and an adjacent portion.

FIG. 7B is a schematic side view of an embodiment of the disclosed embodiments, showing a state where the reinforcing body of the adjacent portion is being fused.

FIG. 7C is a schematic side view of an embodiment of the disclosed embodiments, showing a state where the reinforcing body on the distal end side of the joint part has been removed.

FIG. 7D is a schematic side view of an embodiment of the disclosed embodiments, showing a state where the tubular body and the covering member have been welded together.

FIG. 8 is an enlarged schematic side view showing a part of an embodiment of the disclosed embodiments.

FIG. 9 is an enlarged schematic side view showing a part of an embodiment of the disclosed embodiments.

### DETAILED DESCRIPTION

Hereinafter, some embodiments of the disclosed embodiments will be described with reference to the drawings, however the disclosed embodiments are not limited only to the embodiments below. In each drawing, the covering member is illustrated by a dashed line for convenience. Furthermore, the dimensions of the catheter illustrated in each drawing are dimensions indicated to facilitate the understanding of the contents of the implementation and do not necessarily correspond to the actual dimensions. Also, in each drawing, the left side of the drawing is the distal end side (farther side) to be inserted into the body, and the right side is the proximal side (nearer side, hand side) to be operated by an operator such as a surgeon.

Note that, in the present specification, the “distal end side” is a direction along the longitudinal axis direction of the catheter, and means the direction in which the catheter is inserted deeper into the body. Furthermore, the “proximal end side” is a direction along the longitudinal axis direction of the catheter, and means the opposite direction to the distal end side. Moreover, the “distal end portion” means a site of any member or part that includes the distal end, and extends partway in the longitudinal axis direction from the distal end toward the proximal end side.

<Catheter>

The catheter of the present disclosure includes a tubular body having a lumen, a reinforcing body provided at an outer periphery of the tubular body, and a radiopaque marker member disposed on the reinforcing body along a circumferential direction of the reinforcing body. In a longitudinal axis direction of the tubular body, a distal end of the reinforcing body is either positioned at the same site as a distal end of the marker member, or positioned at a site further toward a distal end side than the distal end of the marker member, and a distal end portion of the marker member is joined to the reinforcing body, and a site of the marker member excluding the distal end portion of the marker member is not joined to the reinforcing body. The details of the catheter of the present disclosure will be described below.

FIG. 1 is a schematic side view showing a catheter 1. The catheter 1, as shown in FIG. 1, generally includes a tubular body 11, a reinforcing body 21, a marker member 31, a covering member 41, and a connector 51.

The tubular body 11 is a cylindrical (hollow-shaped) shaft having a lumen. Specifically, the tubular body 11 can be configured, for example, to have a lumen 11h that penetrates through from an opening 11a at the distal end to the proximal end of the tubular body 11. For example, a medical device such as a guide wire is inserted, or a fluid such as a drug solution is flowed through the lumen 11h.

Because the tubular body 11 is inserted into a body cavity, the material constituting the tubular body 11 preferably has antithrombogenicity, flexibility and biocompatibility. As the material mentioned above, for example, resin materials such as polyamides, polyamide elastomers, polyolefins, polyesters, polyester elastomers, polyurethanes, silicones, fluororesins, and the like, can be used.

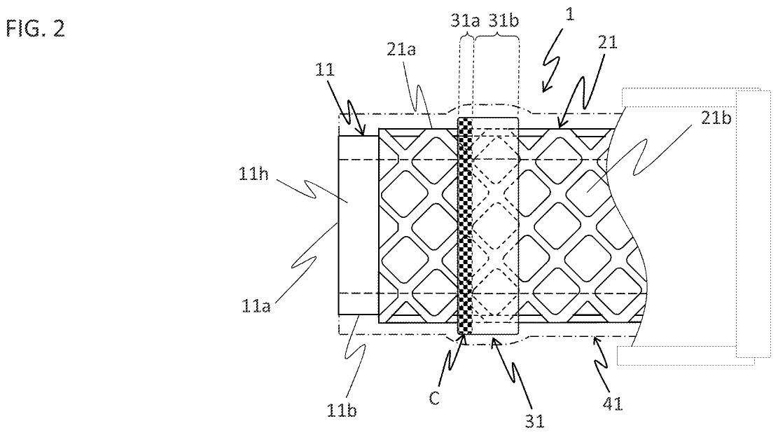

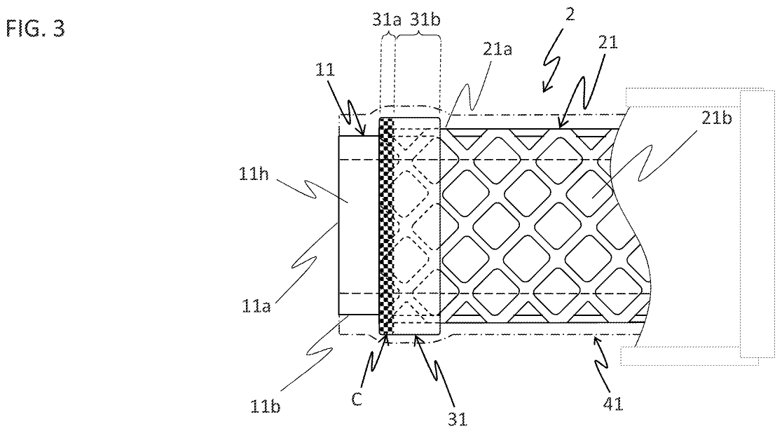

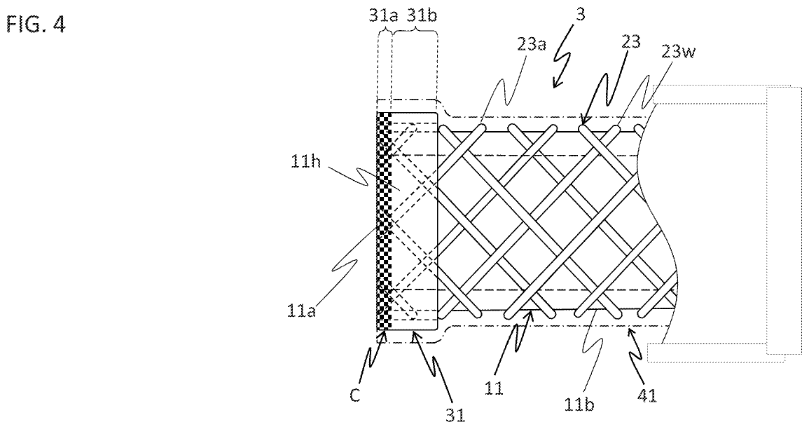

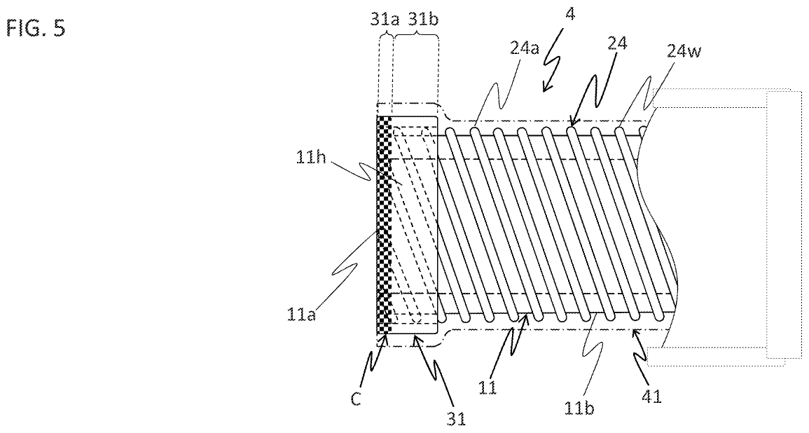

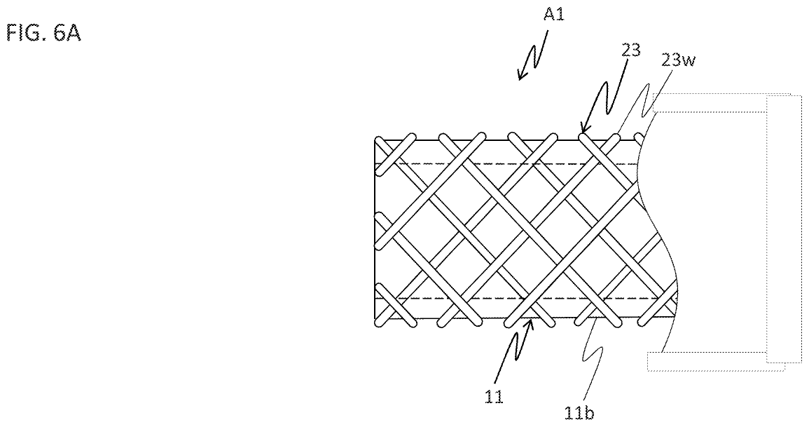

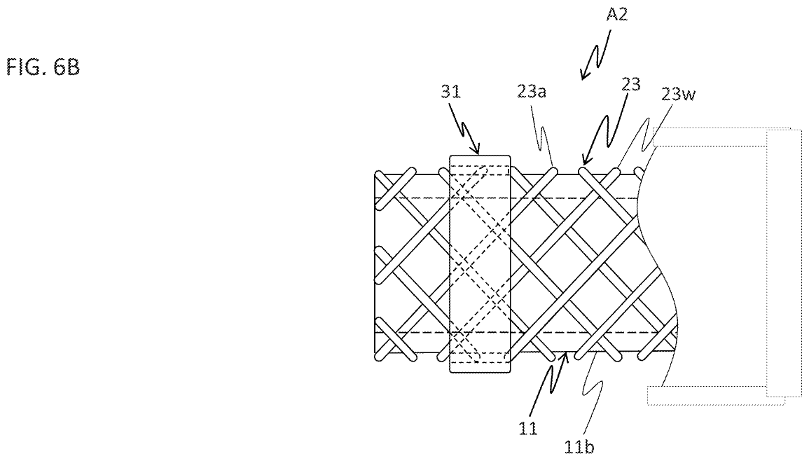

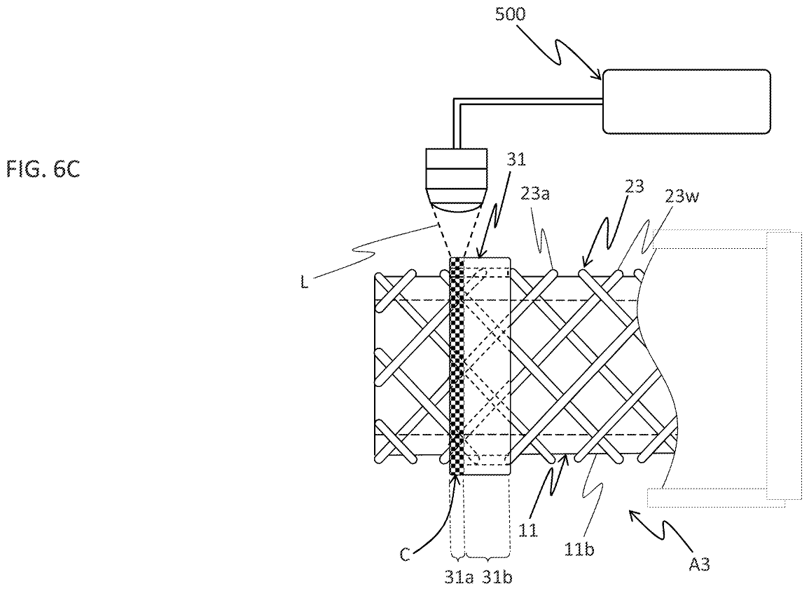

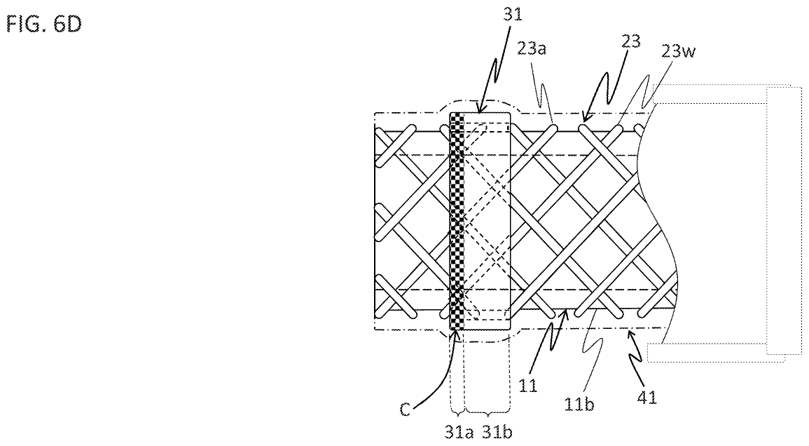

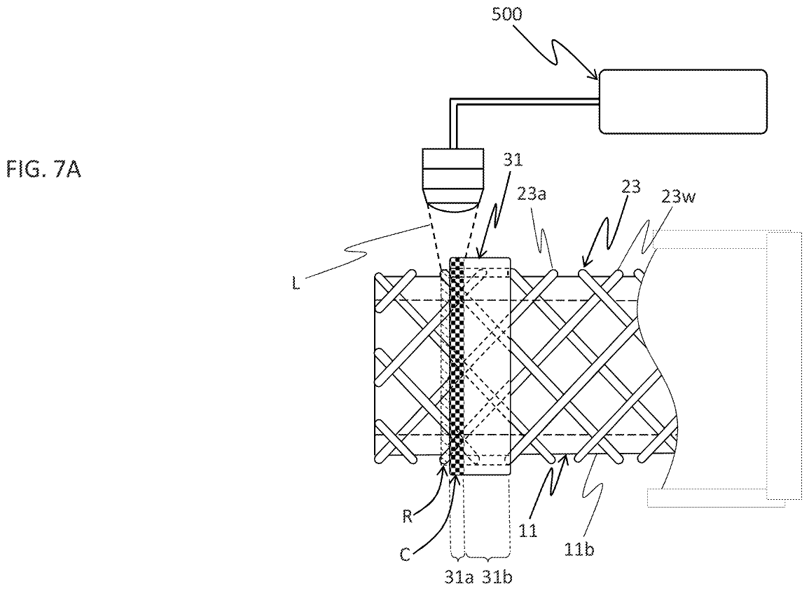

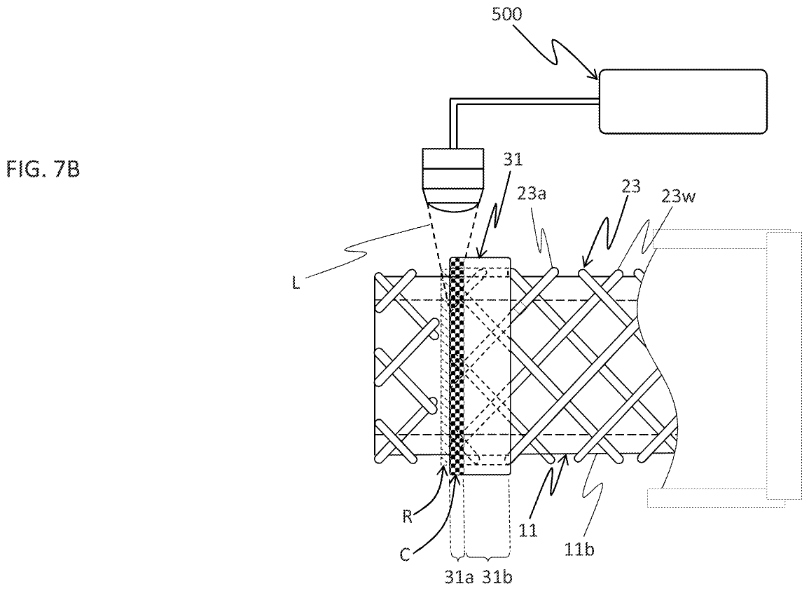

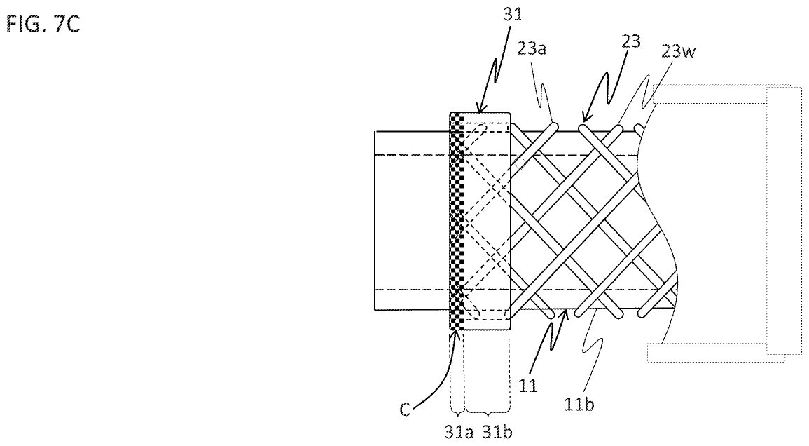

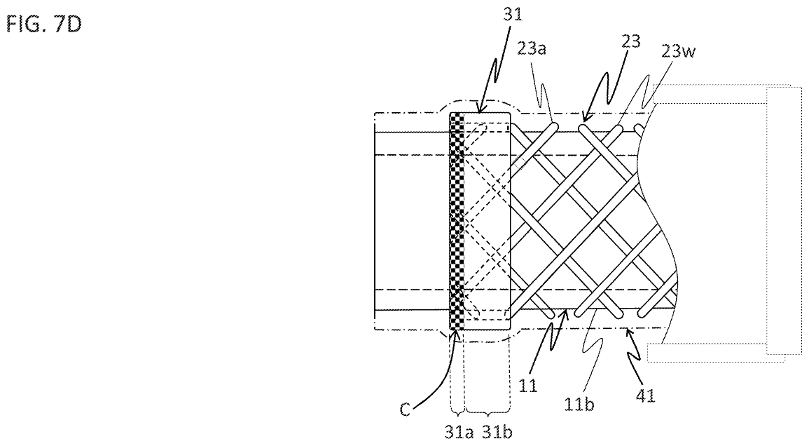

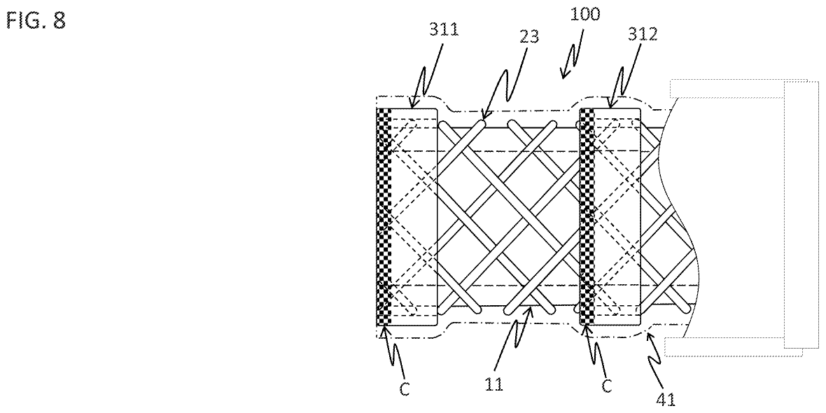

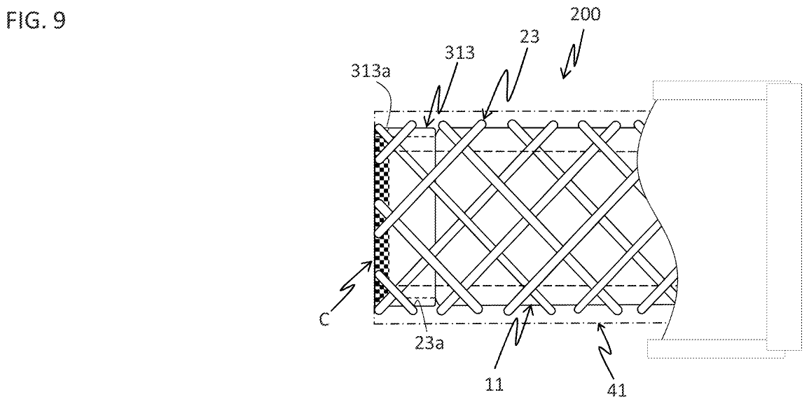

The reinforcing body 21 is a member provided on the outer periphery of the tubular body. As shown in FIG. 2, for example, the reinforcing body 21 is configured by a tubular member, and can be provided so as to make contact with the outer peripheral surface 11b of the tubular body 11 and cover the outer peripheral surface 11b. In the catheter 1, an integrally formed, mesh-like reinforcing body 21 having a large number of openings 21b on the side portion is illustrated. Note that the openings 21b can be formed, for example, by punching the side portion of the cylindrical member with laser light or the like.

Examples of the material constituting the reinforcing body 21 include stainless steel (such as SUS304 and SUS316), metal materials such as tungsten, and resin materials such as reinforced plastics.

The marker member 31 is a radiopaque member disposed on the reinforcing body 21 along the circumferential direction of the reinforcing body 21. Specifically, the marker member 31 can be configured, for example, by an annular member that makes contact with the outer peripheral surface 21a of the reinforcing body 21 and is disposed along the circumferential direction of the reinforcing body 21.

Examples of radiation that is impermeable to the marker member 31 include X-rays, y-rays, and particle beams.

Examples of the material constituting the marker member 31 include radiopaque materials such as gold, platinum, tungsten, and alloys containing these elements (for example, platinum-nickel alloy). The material of the marker member 31 may be a combination of a radiopaque and non-radiopaque material, such as a radiopaque material coated on a surface of a non-radiopaque material, a radiopaque material kneaded in a non-radiopaque resin material, or the like.

Here, the catheter can be configured such that, in the longitudinal axis direction of the tubular body, the distal end of the reinforcing body is either positioned at the same site as the distal end of the marker member, or positioned at a site further toward the distal end side than the distal end of the marker member. Furthermore, the position of the marker member 31 on the reinforcing body 21 can be appropriately selected so as to correspond to the site of the catheter whose position is to be identified by a radiographic image.

In the catheter 1, the distal end of the reinforcing body 21 is located at a site further toward the distal end side than the distal end of the marker member 31 (see FIG. 2).

Moreover, in the present embodiment, the distal end portion of the marker member 31 is joined to the reinforcing body 21, and a site of the marker member 31 excluding the distal end portion of the marker member 31 is not joined to the reinforcing body 21. That is, in the longitudinal axis direction of the tubular body 11, only the distal end portion 31a (distal end) of the marker member 31 is fixed to the reinforcing body 21 (see joint part C in FIG. 2), and a site 31b further toward the proximal end side than the distal end portion 31a of the marker member 31 is not fixed to the reinforcing body 21. The site 31b mentioned above on the proximal end side may be making contact with the reinforcing body 21 (see FIG. 2), or may be spaced apart from the reinforcing body 21.

Examples of the method of joining the marker member 31 and the reinforcing body 21 include, as described in detail in the <Production Method of Catheter> section below, a method of performing welding by irradiating the vicinity of the distal end portion 31a of the marker member 31 with a laser light L.

The covering member 41 is a member that covers the outer periphery of the tubular body 11, the reinforcing body 21, and the marker member 31. Specifically, the covering member 41 can be made of, for example, a soft and highly slidable material, and can be formed having a smooth surface.

Examples of the material constituting the covering member 41 include polyamides, polyamide elastomers, polyesters, and polyurethanes.

As a result of providing the covering member 41 in this manner, for example, the catheter 1 can smoothly move inside a body cavity such as a blood vessel.

The connector 51 is a part used by an operator to push the catheter 1 into the body and/or perform a rotation operation. The connector 51 has the distal end connected to the proximal end of the tubular body 11 and/or the reinforcing body 21. The connector 51 has a lumen 51h that communicates with the lumen 11h of the tubular body 11 at the distal end and is provided with an opening 51a at the proximal end, and during a procedure, it is possible to insert a medical device such as a guide wire, or introduce a fluid such as a drug solution via the lumen 51h.

Next, an example of the manner in which the catheter 1 is used will be described. Here, the catheter 1 is used as a guiding catheter to illustrate a technique in which a balloon catheter is used to dilate a constricted part that has formed in a coronary artery of the heart.

Prior to using the catheter 1, a guide wire A is firstly inserted into the blood vessel, and the distal end of the guide wire A is delivered near the entrance of a coronary artery of the heart. Then, the proximal end portion of the guide wire A is inserted into the lumens 11h and 51h via the opening 11a, and the catheter 1 is pushed forward along the guide wire A into the blood vessel such that the distal end of the catheter 1 reaches the entrance of the coronary artery of the heart. At this time, the catheter 1 is pushed in while confirming the position of the marker member 31 in the blood vessel displayed on a radiographic image display device, and moves forward while following the curvature of the blood vessel due to the flexibility of the catheter 1 (flexibility of the catheter 1 at the attachment part of the marker member 31).

Next, the guide wire A is pulled out and replaced with a thinner guide wire B for a balloon catheter, and the distal end of the guide wire B is made to reach a position via the lumens 51h and 11h where it is passed through the constricted part. Then, a balloon catheter is inserted along the guide wire B to the inside of the constricted part, and the treatment is performed by expanding the constricted part using the balloon. After the treatment described above is completed, the procedure is completed by removing the balloon catheter, the guide wire B, and the catheter 1 in this order.

As described above, because the catheter 1 has the above configuration, the marker member 31 can be fixed to the reinforcing body 21 while ensuring adequate flexibility. As a result, even when a blood vessel has a complicated curved shape, the catheter 1 can be smoothly and accurately delivered to the desired site inside a blood vessel while following the shape and without the marker member 31 detaching from the reinforcing body 21 or moving (becoming displaced) with respect to the reinforcing body 21.

FIG. 3 is an enlarged schematic side view showing a part of a catheter 2. The catheter 2, as shown in FIG. 3, generally includes a tubular body 11, a reinforcing body 21, a marker member 31, a covering member 41, and a connector 51. The catheter 2 differs from the catheter 1 in the positional relationship between the tubular body 11, the reinforcing body 21, and the marker member 31. Because the configurations of the tubular body 11, the reinforcing body 21 and the marker member 31 themselves, and the configurations of the covering member 41 and the connector 51 are the same as those of the catheter 1, the same parts are designated by the same reference numerals and detailed description thereof will not be repeated. Furthermore, the manner in which the catheter 2 is used is the same as that of the catheter 1, and description thereof will not be repeated.

In the catheter 2, the distal end of the reinforcing body 21 is positioned at the same site as the distal end of the marker member 31 in the longitudinal axis direction of the tubular body 11. That is, the position of the distal end of the reinforcing body 21 and the position of the distal end of the marker member 31 are aligned in the longitudinal axis direction, and the position of the distal end of the marker member 31 that is imaged in the radiographic image is configured to match the position of the distal end of the reinforcing body 21.

As described above, because the catheter 2 has the above configuration, the position of the distal end of the reinforcing body 21 can be accurately grasped by referring to the position of the distal end of the marker member 31. As a result, operation of the catheter, whose rigidity generally has a tendency to change before and after the distal end of the reinforcing body in the longitudinal axis direction, can be performed reliably while grasping the position of the catheter.

FIG. 4 is an enlarged schematic side view showing a part of a catheter 3. The catheter 3, as shown in FIG. 4, generally includes a tubular body 11, a reinforcing body 23, a marker member 31, a covering member 41, and a connector 51. The catheter 3 differs from the catheter 2 in the configuration of the reinforcing body 23. Because the configurations of the tubular body 11 and the marker member 31 themselves, and the configurations of the covering member 41 and the connector 51 are the same as those of the catheter 1, the same parts are designated by the same reference numerals and detailed description thereof will not be repeated. Furthermore, the manner in which the catheter 3 is used is the same as that of the catheter 1, and description thereof will not be repeated.

The reinforcing body 23 is a member provided on the outer periphery of the tubular body 11. In the catheter 3, the reinforcing body 23 is provided on the outer peripheral surface of the tubular body 11, and is formed as a braided body (hereinafter, also referred to as “braided body 23”) which is braided using a wire 23w. The braided body 23 can be formed, for example, by using one or more solid wires or twisted wires as the wire 23w, followed by braiding into a mesh form. Note that, a solid wire refers to a single wire, and a twisted wire refers to a bundled group of wires formed by twisting together a plurality of single wires in advance.

As the material of the wire 23w constituting the braided body 23, for example, it is possible to use the same material as the material of the reinforcing body 21 illustrated in the first embodiment and the like.

As described above, because the catheter 3 has the above configuration, the reinforcing body 23 (braided body 23) can be easily formed using the wire 23w.

FIG. 5 is an enlarged schematic side view showing a part of a catheter 4. The catheter 4, as shown in FIG. 5, generally includes a tubular body 11, a reinforcing body 24, a marker member 31, a covering member 41, and a connector 51. The catheter 4 differs from the catheter 3 in the configuration of the reinforcing body 24. Because the configurations of the tubular body 11 and the marker member 31 themselves, and the configurations of the covering member 41 and the connector 51 are the same as those of the catheter 1, the same parts are designated by the same reference numerals and detailed description thereof will not be repeated. Furthermore, the manner in which the catheter 4 is used is the same as that of the catheter 1, and description thereof will not be repeated.

The reinforcing body 24 is a member provided on the outer periphery of the tubular body 11. In the catheter 4, the reinforcing body 24 is provided on the outer peripheral surface 11b of the tubular body 11, and is formed as a coil body that is spirally wound such that a wire 24w surrounds the outer peripheral surface 11b along the longitudinal axis direction. The reinforcing body 24 can be formed, for example, by using one or more solid wires or twisted wires as the wire 24w, and then forming a single-threaded or multi-threaded winding.

As the wire 24w constituting the reinforcing body 24, for example, it is possible to use the same wire 23w constituting the reinforcing body 23 (braided body 23) illustrated in the catheter 3 and the like.

As described above, because the catheter 4 has the above configuration, the reinforcing body 24 can be easily formed using the wire 24w.

<Production Method of Catheter>

The production method of a catheter of the present disclosure is a production method of a catheter provided with a tubular body having a lumen, a reinforcing body provided on an outer periphery of the tubular body, and a radiopaque marker member disposed on the reinforcing body along a circumferential direction of the reinforcing body. The method comprises disposing, in a longitudinal axis direction of the tubular body, a distal end of the reinforcing body at either the same site as a distal end of the marker member, or at a site further toward a distal end side than the distal end of the marker member, and welding a contact portion between the reinforcing body and the distal end portion of the marker member without applying a mechanical external force onto the contact portion, and without welding a site of the marker member excluding the distal end portion of the marker member. An embodiment of the production method of a catheter of the present disclosure will be described below.

For example, the production method of a catheter of the present disclosure can be configured by [Step A] to [Step D] described below. In the present embodiment, a production method of a catheter using the braided body 23 will be illustrated.

[Step A]

Step A is a step for enclosing a tubular body 11 formed in advance with a reinforcing body 23. In the present step, for example, a tubular (cylindrical) braided body 23 (reinforcing body 23) which is braided into a mesh form using a metallic wire 23w is used, and the braided body 23 is made to cover the outer peripheral surface 11b of the tubular body 11 made of resin, and enclose the outer peripheral surface 11b so as to make contact thereto (see the intermediate assembly A1 in FIG. 6A). Note that the braided body 23 may be braided into a tubular shape in advance, or may be formed by winding the wire 23w around the tubular body 11.

[Step B]

Step B is a step for disposing a radiopaque marker member along the circumferential direction on the braided body 23 of the intermediate assembly A1 obtained in Step A. In the present step, for example, an annular and radiopaque metallic marker member 31 is used, and the marker member 31 is disposed making contact with the outer periphery 23a of the braided body 23 and along the circumferential direction thereof, such that the distal end of the braided body 23 is positioned at a site further toward the distal end side than the distal end of the marker member 31 in the longitudinal axis direction of the tubular body 11 (see the intermediate assembly A2 in FIG. 6B). The position of the marker member 31 on the braided body 23 in the longitudinal axis direction can be appropriately disposed so as to correspond to the desired position of the catheter to be identified by the marker member 31.

[Step C]

Step C is a step for welding by heating a contact portion between the braided body 23 and the distal end portion of the marker member 31 (the site at which the joint part C between the braided body 23 and the distal end portion 31a of the marker member 31 is formed) of the intermediate assembly A2 obtained in Step B (see FIG. 6C). In the present step, at the time of the welding described above, the joint part C is formed by welding the contact portion while simply bringing the braided body 23 and the distal end portion 31a of the marker member 31 into contact without applying a mechanical external force. Furthermore, in the production method of a catheter of the present disclosure, a site 31b, the proximal end portion of the marker member 31, is not welded to the braided body 23 (see the intermediate assembly A3 in FIG. 6C). That is, the braided body 23 and the marker member 31 are joined at only the distal end portion 31a of the marker member 31, and the site 31b excluding the distal end portion of the marker member 31 is formed so as to simply make contact with the braided body 23 or be separated from the braided body 23.

Examples of the heating method of the contact portion described above include a method of heating by irradiating a laser light L generated by a laser light generator 500 (see FIG. 6C), and a method of heating by irradiating an electron beam. When heating using a laser light L, it is possible to reliably heat (weld) only a predetermined site without applying a mechanical external force to the irradiated site (in particular, the site forming the joint part C).

[Step D]

Step D is a step for covering the outer periphery of the intermediate assembly A3 obtained in Step C with the covering member 41. In the present step, for example, a tube-shaped covering member is placed on the outer periphery of the intermediate assembly A3, which is then heated in a heating furnace to soften the resin constituting the tubular body 11 and the covering member 41. Then, after removal from the heating furnace and cooling, the resin of the tubular body 11 and the covering member 41 become solidified, and the tubular body 11, the reinforcing body 21, the marker member 31, and the covering member 41 are integrally fixed (see FIG. 6D). The heating temperature, heating time, and atmosphere of the heating furnace can be appropriately selected according to the physical properties, shape, and the like of the resin.

In the production method of a catheter in which the distal end of the braided body 23 and the distal end of the marker member 31 are at the same site in the longitudinal axis direction of the tubular body 11, when the marker member 31 is disposed on the braided body 23, instead of the arrangement of the braided body 23 and the marker member 31 in Step C described above, it is possible for the distal end of the braided body 23 to be disposed at the same site as the distal end of the marker member 31 in the longitudinal axis direction, and for the contact portion to then be welded to form the joint part C.

Furthermore, it is possible for the distal end of the braided body 23 to be disposed at a site further toward the distal end side than the distal end of the marker member 31 in the longitudinal axis direction of the tubular body 11, and at the same time as welding the contact portion by heating, a site of the braided body 23 further toward the distal end side than the marker member 31 may be fused and detached from the contact portion (joint part C). In this case, for example, it is possible to simultaneously irradiate laser light (see FIG. 7A) with respect to the contact portion C and a site of the braided body adjacent to the contact portion on which the marker member is not disposed (adjacent portion R) to weld and detach the braided body 23 (see FIG. 7B), and then remove the site on the distal end side of the braided body 23 that has been detached (see FIG. 7C) and cover it with the covering member 41 (see FIG. 7D). Such a production method enables a catheter in which the position of the distal end of the braided body 23 and the position of the distal end of the marker member 31 are aligned to be easily formed.

As described above, because the production method of the catheter has the above configuration, the marker member 31 can be fixed to the braided body 23 (reinforcing body 23) while ensuring adequate flexibility. Furthermore, deformation of the marker member 31 and the braided body 23 can be suppressed because no mechanical external force is applied to the contact portion (joint part C). Therefore, the lumen 11h of the tubular body 11 is not unintentionally narrowed, and the outermost peripheral surface of the catheter does not bulge, which enables a catheter that can be smoothly operated to be obtained.

Note that the present disclosure is not limited to the configurations of the embodiments described above, but is stipulated by the claims, and the present disclosure is intended to include all modifications within the meaning and scope equivalent to the claims. Part of the configurations of the embodiments described above may be deleted or replaced with another configuration, and other configurations may be added to the configurations of the embodiments described above.

For example, in the catheters 1 to 4 described above, a single marker member 31 is disposed on the reinforcing bodies 21, 23 and 24, and the production methods therefor, have been described. However, as shown in FIG. 8, a catheter 100 is also possible in which two or more marker members (for example, a first marker member 311 and a second marker member 312) are disposed in the longitudinal axis direction. This allows a plurality of sites of the catheter 100 to be identified, and the procedure to be more accurately performed.

Furthermore, in the catheters 1 to 4 described above, the marker member 31 making contact with the outer periphery of the reinforcing bodies 21, 23 and 24 has been described. However, for example, as shown in FIG. 9, a catheter 200 is also possible in which a marker member 313 makes contact with the inner periphery 23a of the reinforcing body 23.

Moreover, in the catheters 1 to 4 described above, the catheters having the annular marker member 31 have been described. However, the marker member need only be disposed on the reinforcing body along the circumferential direction of the reinforcing body, and the shape of the marker member may be a shape other than an annular shape, such as a flat plate shape, or an arc shape along the circumferential direction of the reinforcing body.

In addition, in the embodiment of the production method of a catheter described above, the production method of a catheter provided with the braided body 23 (reinforcing body 23) which is braided using the metallic wire 23w has been described. However, the material constituting the reinforcing body is not particularly limited as long as the reinforcing body and the marker member can be welded together. The reinforcing body may be formed of a resin material or a material in which a resin and a metal have been combined.

1. A catheter comprising:
a tubular body having a lumen;
a reinforcing body provided on an outer periphery of the tubular body; and
a radiopaque marker member disposed on the reinforcing body along a circumferential direction of the reinforcing body, wherein
in a longitudinal axis direction of the tubular body, a distal end of the reinforcing body is either positioned at the same site as a distal end of the marker member, or positioned at a site further toward a distal end side of the tubular body than the distal end of the marker member, and
a distal end portion of the marker member is joined to the reinforcing body, and a portion of the marker member other than the distal end portion of the marker member is not joined to the reinforcing body.

2. The catheter according to claim 1, wherein
in the longitudinal axis direction of the tubular body, the distal end of the reinforcing body is positioned at the same site as the distal end of the marker member.

3. The catheter according to claim 1, wherein
the reinforcing body is a braided body that is braided using a wire.

4. The catheter according to claim 1, wherein
the distal end portion of the marker member that is joined to the reinforcing body extends for a first dimension in the longitudinal axis direction of the tubular body that is smaller than a second dimension for which the portion of the marker member other than the distal end portion extends in the longitudinal axis direction of the tubular body.

5. The catheter according to claim 1, wherein
the portion of the marker member other than the distal end portion is either in contact with the reinforcing body or spaced apart from the reinforcing body.

6. A production method of a catheter provided with a tubular body having a lumen, a reinforcing body provided on an outer periphery of the tubular body, and a radiopaque marker member disposed on the reinforcing body along a circumferential direction of the reinforcing body, the method comprising:
disposing, in a longitudinal axis direction of the tubular body, a distal end of the reinforcing body at either the same site as a distal end of the marker member, or at a site further toward a distal end side of the tubular body than the distal end of the marker member; and
welding a contact portion formed by the reinforcing body and the distal end portion of the marker member without applying a mechanical external force to the contact portion, and without welding a portion of the marker member other than the distal end portion of the marker member.

7. The production method according to claim 6, wherein
in the longitudinal axis direction of the tubular body, the distal end of the reinforcing body is disposed at the site further toward the distal end side than the distal end of the marker member, and
at the same time as welding the contact portion by heating, a site of the reinforcing body further toward the distal end side than the distal end of the marker member is detached from the contact portion.

8. The production method according to claim 6, wherein
heating of the contact portion during welding is performed by irradiation of laser light.
